SEQUENCE LISTING (5 sequences)

SEQ ID NO: 1 - protein, 225 aa, Homo sapiens
GFLITRHSQTTDAPQCPQGTLQVYEGFSLLYVQGNKRAHGQDLGTAGSCLRRFSTMPFMFCNINNVCNFASRNDYSYWLSTPEPMPMSMQPLKGQSIQPFISRCAVCEAPAVVIAVHSQTIQIPHCPQGWDSLWIGYSFMMHTSAGAEGSGQALASPGSCLEEFRSAPFIECHGRGTCNYYANSYSFWLATVDVSDMFSKPQSETLKAGDLRTRISRCQVCMKRT

SEQ ID NO: 2 - DNA, 677 bp, Homo sapiens
ggatttcttattacacgccacagccagacaacggatgcaccacaatgcccacagggaacacttcaggtctatgaaggcttttctctcctgtatgtacaaggaaataaaagagcccacggtcaagacttggggacggctggcagctgccttcgtcgctttagtaccatgcctttcatgttctgcaacatcaataatgtttgcaactttgcttcaagaaatgactattcttactggctctctaccccagagcccatgccaatgagcatgcaacccctaaagggccagagcatccagccattcattagtcgatgtgcagtatgtgaagctccagctgtggtgatcgcagttcacagtcagacgatccagattccccattgtcctcagggatgggattctctgtggattggttattccttcatgatgcatacaagtgcaggggcagaaggctcaggtcaagccctagcctcccctggttcctgcttggaagagtttcgttcagctcccttcatcgaatgtcatgggaggggtacctgtaactactatgccaactcctacagcttttggctggcaactgtagatgtgtcagacatgttcagtaaacctcagtcagaaacgctgaaagcaggagacttgaggacacgaattagccgatgtcaagtgtgcatgaagaggacata

SEQ ID NO: 3 - protein, 226 aa, Homo sapiens
GFVFTRHSQTTAIPSCPEGTVPLYSGFSFLFVQGNQRAHGQDLGTLGSCLQRFTTMPFLFCNVNDVCNFASRNDYSYWLSTPALMPMNMAPITGRALEPYISRCTVCEGPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKRH

SEQ ID NO: 4 - DNA, 731 bp, Homo sapiens
ggtttgaaaggaaaacgtggagacagtggatcacctgcaacctggacaacgagaggctttgtcttcacccgacacagtcaaaccacagcaattccttcatgtccagaggggacagtgccactctacagtgggttttcttttctttttgtacaaggaaatcaacgagcccacggacaagaccttggaactcttggcagctgcctgcagcgatttaccacaatgccattcttattctgcaatgtcaatgatgtatgtaattttgcatctcgaaatgattattcatactggctgtcaacaccagctctgatgccaatgaacatggctcccattactggcagagcccttgagccttatataagcagatgcactgtttgtgaaggtcctgcgatcgccatagccgttcacagccaaaccactgacattcctccatgtcctcacggctggatttctctctggaaaggattttcattcatcatgttcacaagtgcaggttctgagggcaccgggcaagcactggcctcccctggctcctgcctggaagaattccgagccagcccatttctagaatgtcatggaagaggaacgtgcaactactattcaaattcctacagtttctggctggcttcattaaacccagaaagaatgttcagaaagcctattccatcaactgtgaaagctggggaattagaaaaaataataagtcgctgtcaggtgtgcatgaagaaaagaca

SEQ ID NO: 5 - protein, 17 aa, Homo sapiens
VCNFASRNDYSYWLSTP

(19) United States
(12) Patent Application Publication
(10) Pub. No.: US 2012/0190612 A1
(43) Pub. Date: Jul. 26, 2012

(54) METHODS OF TREATMENT

(75) Inventors: Judith Lee Black, Woollahra (AU); Janette Kay Burgess, Wilberforce (AU); Brian Gregory George Oliver, Dundas Valley (AU); Markus Weckmann, Muhlbach/Kiefersfelden (DE)

(73) Assignee: CRC FOR ASTHMA AND AIRWAYS LTD., GLEBE, AU (unknown)

(21) Appl. No.: 13/376,939

(22) PCT Filed: Jun. 9, 2010
(86) PCT No.: PCT/AU10/00710

§ 371 Date: Feb. 22, 2012

(30) Foreign Application Priority Data
Jun. 9, 2009 (AU) 2009902633
Oct. 21, 2009 (AU) 2009905173

(51) Int. Cl. A61K 38/17; A61K 38/02; A61K 38/10; A61P 37/06; A61K 31/7088; A61P 35/04; A61P 19/02; A61P 11/06; C12N 5/071; A61P 9/00

(52) U.S. Cl. 514/1.7; 435/375; 514/1.1; 514/21.4; 514/13.3; 514/44 R; 514/19.8; 514/16.6

(57) ABSTRACT

The present invention relates to methods for inhibiting proliferation and/or migration of lymphatic endothelial cells and for inhibiting lymphangiogenesis, comprising administering to a subject, or to lymphatic endothelial cells derived therefrom, an effective amount of a collagen type IV-derived NC1 domain polypeptide or a fragment, derivative or variant thereof, a polynucleotide encoding the same, or an agent capable of increasing the expression or production of the NC1 domain polypeptide. Aso provided are methods for the treatment or prevention of diseases and conditions associated with aberrant lymphatic endothelial cell activity.

### FIELD OF THE INVENTION

The present invention relates generally to methods for modulating the activity of cells of the lymphatic system, in particular lymphatic endothelial cells. More specifically, the invention relates to methods for inhibiting proliferation and/or migration of lymphatic endothelial cells and for inhibiting lymphangiogenesis, including tumour-induced lymphangiogenesis. Accordingly, embodiments of the invention relate to the treatment of diseases and conditions associated with aberrant lymphatic endothelial cell activity.

### BACKGROUND OF THE INVENTION

The disclosure of every patent, patent application, and publication cited herein is hereby incorporated herein by reference in its entirety.

The citation of any reference herein should not be construed as an admission that such reference is available as “Prior Art” to the present application. Further, the reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Cancer can arise in any organ of the body. In many instances if the neoplasm is confined to its organ of origin, the cancer can often be managed and treated effectively, and potentially the patient cured, through existing therapies and/or surgical removal of the tumour mass. Unfortunately, many cancers spread or metastasize to other sites in the body, and cancer metastasis is the leading cause of death in cancer patients. Determination of the mechanisms and routes by which cancer metastasis occurs is clearly of significant importance in the identification and development of novel therapeutic targets for anti-cancer treatment.

Cancer cells can spread within the body by different mechanisms, such as direct invasion of surrounding tissues, spread via the bloodstream (hematogenous metastasis) and spread via the lymphatic system (lymphatic metastasis). In the case of solid tumours, metastasis to local lymph nodes via the lymphatic vessels is a common step in their spread. Despite its clinical relevance, surprisingly little is known about the mechanisms leading to metastasis via the lymphatic system.

It is now known that a variety of cancers, in particular solid tumours are able to activate or induce lymphangiogenesis, the formation of new vessels of the lymphatic system. For example the growth factors VEGF-C and VEGF-D have been shown to promote lymphangiogenesis and lymphatic metastasis in tumours (Stacker et al. Nature Med. 7, 186-191, 2001). Tumour-induced lymphangiogenesis may promote the spread of tumours to lymph nodes. The spread of a tumour to the lymph nodes is an important prognostic indicator in many types of cancer and is the basis for surgical and radiation treatments of draining regional lymph nodes in an attempt to combat the disease. However once a tumour has spread to the lymph nodes, there is often little that can be done to resolve the cancer entirely. It would be preferable to minimise or abolish the potential for the spread of the cancer, and thus modulation of lymphangiogenesis represents a particularly attractive target for anti-cancer agents.

Lymphatic vasculature also plays a critical role in immunity and maintaining interstitial fluid homeostasis. Lymph fluid accumulation results from changes to normal lymphatic function. In addition to its induction by tumours, lymphangiogenesis is also implicated in a variety of other disorders such as oedema, pulmonary fibrosis, rheumatoid arthritis, asthma, transplant rejection, psoriasis and impaired wound repair. Moreover abnormal lymphatic system function can give rise to tumours such as lymphangioma and Kaposi's sarcoma.

There remains a need for effective therapeutic and prophylactic options for the treatment of diseases and conditions associated with abnormal lymphangiogenesis and/or aberrant lymphatic endothelial cell activity.

### SUMMARY OF THE INVENTION

The present invention is predicated upon the inventors' surprising finding that the NC1 domain of the α5 chain of collagen type IV (herein also referred to as lamstatin) and NC1 domain of the α3 chain of collagen type IV (also known as tumstatin) have surprising activity in lymphatic endothelial cells. Cell proliferation and migration is inhibited in the presence of lamstatin and tumstatin. Significantly these polypeptides also inhibit new lymphatic vessel formation.

According to a first aspect of the invention there is provided a method for inhibiting proliferation and/or migration of lymphatic endothelial cells in a subject, the method comprising administering to the subject, or to lymphatic endothelial cells derived therefrom, an effective amount of a collagen type IV-derived NC1 domain polypeptide or a fragment, derivative or variant thereof, a polynucleotide encoding the same, or an agent capable of increasing the expression or production of the NC1 domain polypeptide.

In an embodiment the collagen type IV-derived NC1 domain polypeptide may be the NC1 domain of the α5 chain of collagen type IV (lamstatin). The lamstatin may comprise an amino acid sequence as set forth in SEQ ID NO:1. The lamstatin may be encoded by a polynucleotide comprising a nucleotide sequence as set forth in SEQ ID NO:2.

In an embodiment the collagen type IV-derived NC1 domain polypeptide may be the NC1 domain of the α3 chain of collagen type IV (tumstatin). The tumstatin may comprise an amino acid sequence as set forth in SEQ ID NO:3. The tumstatin may be encoded by a polynucleotide comprising a nucleotide sequence as set forth in SEQ ID NO:4.

The fragment may comprise the amino acid sequence VCNFASRNDYSYWLSTP (SEQ ID NO:5) or a variant thereof.

According to a second aspect of the invention there is provided a method for inhibiting lymphangiogenesis, the method comprising administering to a subject, or cells or lymphatic tissue derived therefrom, a collagen type IV-derived NC1 domain polypeptide or a fragment, derivative or variant thereof, a polynucleotide encoding the same, or an agent capable of increasing the expression or production of the NC1 domain polypeptide.

The lymphangiogenesis may be tumour-induced lymphangiogenesis. In an exemplary embodiment the tumour is a lung adenocarcinoma. Cancer metastasis may be reduced following the administration step.

According to a third aspect of the invention there is provided a method for the treatment or prevention of a disease or condition associated with aberrant lymphatic endothelial cell activity, the method comprising administering to the subject, or to lymphatic endothelial cells derived therefrom, an effective amount of a collagen type IV-derived NC1 domain polypeptide or a fragment, derivative or variant thereof, a polynucleotide encoding the same, or an agent capable of increasing the expression or production of the NC1 domain polypeptide.

The disease or condition may be associated with abnormal, excessive or otherwise aberrantly regulated lymphangiogenesis. The disease or condition may be selected from, for example, tumour-induced lymphangiogenesis, cancer metastasis, fibrosis, lymphangioleiomyomatosis (LAM), rheumatoid arthritis, asthma, transplant rejection, psoriasis, impaired wound repair, lymphangioma or Kaposi's sarcoma. The fibrosis may be pulmonary fibrosis.

According to a fourth aspect of the invention there is provided the use of a collagen type IV-derived NC1 domain polypeptide or a fragment, derivative or variant thereof, a polynucleotide encoding the same, or an agent capable of increasing the expression or production of the NC1 domain polypeptide, for the inhibition of lymphatic endothelial cell proliferation and/or migration, lymphangiogenesis, or treatment or prevention of a disease or condition associated with aberrant lymphatic endothelial cell activity.

According to a fifth aspect of the invention there is provided the use of a collagen type IV-derived NC1 domain polypeptide or a fragment, derivative or variant thereof, a polynucleotide encoding the same, or an agent capable of increasing the expression or production of the NC1 domain polypeptide, for the manufacture of a medicament for the inhibition of lymphatic endothelial cell proliferation and/or migration, lymphangiogenesis, or treatment or prevention of a disease or condition associated with aberrant lymphatic endothelial cell activity.

Also provided herein are pharmaceutical compositions comprising a collagen type IV-derived NC1 domain polypeptide or a fragment, derivative or variant thereof, a polynucleotide encoding the same, or an agent capable of increasing the expression or production of the NC1 domain polypeptide, optionally together with suitable pharmaceutically acceptable carriers and/or diluents.

According to a sixth aspect of the invention there is provided a method for the promotion of lymphangiogenesis, the method comprising administering to a subject, or cells or lymphatic tissue derived therefrom, an inhibitor of a collagen type IV-derived NC1 domain polypeptide or a fragment, derivative or variant thereof, a polynucleotide encoding the same.

The subject may suffer from a condition characterised by, or otherwise associated with, impaired lymphangiogenesis. The condition may be oedema. The oedema may be lymphoedema, surgery-induced oedema or tumour-induced oedema.

Also provided herein are pharmaceutical compositions comprising an inhibitor of a collagen type IV-derived NC1 domain polypeptide or a fragment, derivative or variant thereof, a polynucleotide encoding the same, optionally together with suitable pharmaceutically acceptable carriers and/or diluents.

### BRIEF DESCRIPTION OF THE DRAWINGS

Embodiments of the invention are described herein, by way of non-limiting example only, with reference to the following drawings.

FIG. 1. Cell specific effect of lamstatin on cell viability. Treatment of (A) fibroblasts (3567), (B) epithelial cells (A549), and (C) lymphatic endothelial cells (HMVEC-LLy) with lamstatin with various concentrations for 72 hrs. Cell viability was measured with MTT. Note the cell activity at the highest concentration, indicating a non-cytotoxic pathway. n=3. *p<0.05.

FIG. 2. A. Anti-proliferative effect of lamstatin on lymphatic endothelial cells. Time (min) required to reach a threshold cell index of 1 (C1>4 maximum recorded after 72 h) is indicated. Cells treated with 10 μg/mL lamstatin required double the amount of time to reach a comparable cell number as in the vehicle control. Linear trend confirmed from ANOVA post tests with p=0.0002. **p<0.01. B. Effect of lamstatin on lymphatic endothelial cell attachment in vitro. Shown is a reduction in cell attachment after 55 min of treatment with lamstatin at various concentrations.

FIG. 3. Tube formation by HMVEC-LLy on Matrigel after 24 hrs in BGM-2 media. Tumstatin (A), lamstatin (B) and the consensus peptide CP17 (C) reduce in a concentration related manner the numbers of tube-like structures formed. CP17 concentration is given in μM, with the highest concentration being equal to ˜25× that of either tumstatin or lamstatin. n=2, Kruskal-Wallis with *p<0.05 and **p<0.05

FIG. 4. Migration of HMVEC-LLy towards a VEGFD gradient after 24 hrs. A. Increasing concentrations of VEGFD attract lymphatic endothelial cells in a concentration related manner. B. Addition of a VEGFD specific blocking fusion receptor (R3-FC) diminished migration from 42.000 RFU to less than 15.000 RFU. Lamstatin was equally potent (10.000 RFU) and tumstatin showed similar tendencies. n=1.

FIG. 5. Binding of integrins β3 (A), β1 (B) and α5 (C) to HMVEC-LLy cells in the presence of lamstatin (10 μg/ml), peptide CP17 (5 μM) or vehicle (1 nM EDTA, pH 3.5). Integrin binding was determined using monoclonal antibodies specific for integrins β3, β1 and α5.

FIG. 6. Treatment of LAM nodule cells with increasing concentrations of lamstatin (A to C) or tumstatin (D to F). Nodule cells were separated according to their location within the nodule; intermediate layer (A and D), centre (B and E) and outer layer (C and F). The maximum reduction of cell viability observed was >50% in nodule cells in the presence of lamstatin and tumstatin. n=6 (3 individual test from different passages).

FIG. 7. Length of tubular structures and number of tubes per square pixel for the treatment of nodule cells with lamstatin for 5 hrs. For tube length (A) 10 tubular structures that were a straight connection between two cells or a cell cluster were measured in every picture. To calculate the number of tubular structures (B) the picture was divided into 9 segments of known area and number of tubes per segment was counted.

FIG. 8. (A) Mean intensity of LyVe-1 staining of whole mount stained ears of tumour-bearing mice treated with 10 or 100 μg/mL lamstatin or vehicle (1 nM EDTA, pH 3.0) only. (B) Mean intensity of PECAM staining of whole mount stained ears of tumour-bearing mice treated with 10 or 100 μg/mL lamstatin or vehicle only.

FIG. 9. Confocal microscope images of whole mount stained ears of mice that received (A) Matrigel only, (B) tumour cells+vehicle only, (C) tumour cells+10 μg/mL lamstatin or (D) tumour cells+100 μg/mL lamstatin. For each set of images, the top left panel shows tumour cells, the top right panel shows staining for PECAM, the bottom left panel shows staining for LyVe-1, and the bottom right panel is an overlay image of the other three panels.

FIG. 10. Lymphatic vessel morphology as measured in whole mount stained ears of tumour-bearing mice treated with 10 or 100 μg/mL lamstatin as compared to vehicle (1 nM EDTA, pH 3.0) only (A and B) or with 5 or 50 μA CP17 as compared to vehicle (1 nM EDTA, pH 3.0) only (C and D). Vessel morphology was determined using Image J software as the number of branches (A and C) or loops (B and D).

The subject specification contains amino acid and nucleotide sequence information prepared using the programme PatentIn Version 3.4, presented herein in a Sequence Listing. Amino acid and polynucleotide sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. Specifically, the amino acid sequence of human lamstatin is provided in SEQ ID NO:1 and the encoding nucleotide sequence is provided in SEQ ID NO:2. The amino acid sequence of human tumstatin is provided in SEQ ID NO:3 and the encoding nucleotide sequence is provided in SEQ ID NO:4. SEQ ID NO:5 provides the amino acid sequence of an exemplary functional peptide fragment of lamstatin and tumstatin.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The articles “a” and “an” are used herein to refer to one or to more than one (i.e., to at least one) of the grammatical object of the article. By way of example, “an element” means one element or more than one element.

The term “abnormal” when used herein in relation to lymphangiogenesis means lymphangiogenesis that is undesirable or inappropriately regulated. Thus abnormal lymphangiogenesis may be upregulated or excessive with respect to normally regulated lymphangiogenesis, or alternatively may be downregulated, impaired or suppressed with respect to normally regulated lymphangiogenesis. In each case the alteration or abnormality in lymphangiogenesis may be quantitative, temporal and/or spatial. That is, in the case of upregulated or excessive lymphangiogenesis for example, lymphangiogenesis may occur at an abnormally high level, occur at a time when lymphangiogenesis would normally not occur, and/or occur in a tissue or location where lymphangiogenesis would normally not occur. Similarly, in the case of impaired or suppressed lymphangiogenesis, a tissue or a body's ability to induce or initiate lymphangiogenesis may be impaired such that lymphangiogenesis cannot occur at sufficient levels, and/or occur in the required circumstances (time and/or location) to maintain a normal healthy state. Those skilled in the art will appreciate that the term “aberrant” in relation to lymphatic endothelial cell activity enjoys a similar scope. Typically “aberrant” activity refers to abnormal, excessive or otherwise unwanted cellular proliferation and/or migration.

In the context of this specification, the term “activity” as it pertains to a protein, polypeptide or polynucleotide means any cellular function, action, effect or influence exerted by the protein, polypeptide or polynucleotide, either by a nucleic acid sequence or fragment thereof, or by the protein or polypeptide itself or any fragment thereof.

As used herein the term “associated with” when used in the context of a disease or condition “associated with” abnormal lymphangiogenesis or aberrant lymphatic endothelial cell activity, means that the disease or condition may result from, result in, be characterised by, or otherwise associated with the abnormal lymphangiogenesis or aberrant lymphatic endothelial cell activity. Thus, the association between the disease or condition and the abnormal lymphangiogenesis or aberrant lymphatic endothelial cell activity may be direct or indirect and may be temporally and/or spatially separated.

As used herein the term “effective amount” includes within its meaning a non-toxic but sufficient amount or dose of an agent or compound to provide the desired effect. The exact amount or dose required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

It will be understood that as used herein the term “expression” may refer to expression of a polypeptide or protein, or to expression of a polynucleotide or gene, depending on the context. The polynucleotide may be coding or non-coding (e.g. miRNA). Expression of a polynucleotide may be determined, for example, by measuring the production of RNA transcript levels. Expression of a protein or polypeptide may be determined, for example, by immunoassay using an antibody(ies) that bind with the polypeptide.

The term “inhibiting” and variations thereof such as “inhibition” and “inhibits” as used herein do not necessarily imply the complete inhibition of the specified event, activity or function. Rather, the inhibition may be to an extent, and/or for a time, sufficient to produce the desired effect. Inhibition may be prevention, retardation, reduction or otherwise hindrance of the event, activity or function. Such inhibition may be in magnitude and/or be temporal in nature. In particular contexts, the terms “inhibit” and “prevent”, and variations thereof may be used interchangeably.

In the context of this specification, the term “inhibitor” refers to any agent or action capable of inhibiting either or both the expression or activity of lamstatin or tumstatin, either directly or indirectly. Accordingly the inhibitor may operate directly or indirectly on the lamstatin or tumstatin polypeptide, the corresponding mRNA or genes, or alternatively act via the direct or indirect inhibition of any one or more components of a lamstatin- or tumstatin-associated pathway. Such components may be molecules activated, inhibited or otherwise modulated prior to, in conjunction with, or as a consequence of lamstatin or tumstatin activity. Thus, the inhibitor may operate to prevent transcription, translation, post-transcriptional or post-translational processing or otherwise inhibit the activity of lamstatin or tumstatin or a component of a lamstatin- or tumstatin-associated pathway in any way, via either direct or indirect action. The inhibitor may for example be nucleic acid, peptide, any other suitable chemical compound or molecule or any combination of these. It will be understood that in indirectly impairing the activity of lamstatin or tumstatin or a component of a lamstatin- or tumstatin-associated pathway, the inhibitor may effect the activity of molecules which regulate, or are themselves subject to regulation or modulation by, lamstatin or tumstatin or a component of a lamstatin- or tumstatin-associated pathway.

As used herein the term “lamstatin” refers to the α5 chain of the non-collagenous (NC1) domain of collagen type IV. The term “tumstatin” refers to the α3 chain of the non-collagenous (NC1) domain of collagen type IV. In each case, the terms “lamstatin” and “tumstatin” typically refer to those polypeptides as found in humans, or to derivative, fragments or variants thereof. However those skilled in the art will appreciate that homologues of human lamstatin and tumstatin from other species are also contemplated and encompassed by the present disclosure.

As used herein the term “polypeptide” means a polymer made up of amino acids linked together by peptide bonds. The terms “polypeptide” and “protein” are used interchangeably herein, although for the purposes of the present invention a “polypeptide” may constitute a portion of a full length protein. The term “polynucleotide” as used herein refers to a single- or double-stranded polymer of deoxyribonucleotide, ribonucleotide bases or known analogues or natural nucleotides, or mixtures thereof. In some contexts in the present specification the terms “polynucleotide” and “nucleic acid molecule” are used interchangeably.

The term “subject” as used herein refers to mammals and includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Typically, the mammal is human or a laboratory test animal. Even more typically, the mammal is a human.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery. In conditions which display or a characterized by multiple symptoms, the treatment or prevention need not necessarily remedy, prevent, hinder, retard, or reverse all of said symptoms, but may prevent, hinder, retard, or reverse one or more of said symptoms.

The collagen type IV family of molecules is comprised of 6 isoforms, encoded in humans in pairs on 3 chromosomes (13q34 for colIVα1 and α2, 2q36.2 for colIVα3 and α4, Xq22.3 for colIVα5 and α6). The collagen IV molecule is made up of a 7s domain, followed by a helical domain and a non-collagenous (NC) domain. In the lung the collagen network is formed by the alignment of 3 collagen IV molecules to a fibril, that connects, via its 7S and NC domains, to other collagen fibrils. It is established that the NC domains of colIVα1 (arresten), colIVα2 (canstatin) and collVα3 (tumstatin) show distinct functionality, once cleaved off from the fibrils. In case of tumstatin, the effect is anti-angiogenic and it induces apoptosis in proliferating endothelial cells. To date understanding of the function of colIVα5 has remained elusive.

As exemplified herein it has been found that the NC1 domains of the α5 and α3 chains of collagen type IV (herein referred to as lamstatin and tumstatin, respectively) have surprising activity in lymphatic endothelial cells. Cell proliferation and migration is inhibited in the presence of lamstatin and tumstatin. Significantly these polypeptides also inhibit lymphatic tube formation. Therefore these findings have enabled the development of methods of inhibiting or reducing the incidence and/or severity of lymphangiogenesis. In the context of cancer treatment for example these findings are extremely valuable in that they provide an adjunct treatment regimen directed to minimising or reducing the occurrence of cancer metastasis. Accordingly, the findings disclosed herein have enabled the design of novel therapeutic and prophylactic methods for reducing the progression and spread of cancer.

One aspect of the present invention is directed to a method for inhibiting proliferation and/or migration of lymphatic endothelial cells in a subject, the method comprising administering to the subject, or to lymphatic endothelial cells derived therefrom, an effective amount of a collagen type IV-derived NC1 domain polypeptide or a fragment, derivative or variant thereof, a polynucleotide encoding the same, or an agent capable of increasing the expression or production of the NC1 domain polypeptide.

Another aspect of the invention provides a method for inhibiting lymphangiogenesis, the method comprising administering to a subject, or cells or lymphatic tissue derived therefrom, a collagen type IV-derived NC1 domain polypeptide or a fragment, derivative or variant thereof, a polynucleotide encoding the same, or an agent capable of increasing the expression or production of the NC1 domain polypeptide.

Another aspect of the invention provides a method for the treatment or prevention of a disease or condition associated with aberrant lymphatic endothelial cell activity, the method comprising administering to the subject, or to lymphatic endothelial cells derived therefrom, an effective amount of a collagen type IV-derived NC1 domain polypeptide or a fragment, derivative or variant thereof, a polynucleotide encoding the same, or an agent capable of increasing the expression or production of the NC1 domain polypeptide.

Embodiments of the invention provide methods for the modulation of lymphangiogenesis by modulating the amount of lamstatin, tumstatin or derivatives, fragments or variants thereof in lymphatic endothelial cells. In circumstances where lymphangiogenesis is excessive or unregulated, increasing the amount and/or activity of lamstatin, tumstatin, or derivatives, fragments or variants thereof, inhibits lymphangiogenesis. In circumstances where lymphangiogenesis is insufficient or suppressed, decreasing the amount and/or activity of lamstatin, tumstatin, or derivatives, fragments or variants thereof, promotes or induces lymphangiogenesis. Increasing or decreasing the amount of lamstatin or tumstatin may be relative to normal endogenous levels. Reference to “normal endogenous levels” should be understood as a reference to the level of lamstatin or tumstatin which is expressed in lymphatic endothelial cells of a subject in which lymphangiogenesis is normally regulated. It would be appreciated by the person of skill in the art that this “normal level” is likely to correspond to a range of levels, as opposed to a singularly uniform discrete level, due to differences between cohorts of individuals. By “cohort” is meant a cohort characterised by one or more features which are also characteristic of the subject who is undergoing treatment. These features include, but are not limited to, age, gender or ethnicity, for example. Accordingly, reference herein to modulating lamstatin levels relative to normal endogenous levels is a reference to increasing or decreasing lamstatin levels relative to either a discrete lamstatin level which may have been determined for normal individuals who are representative of the same cohort as the individual being treated or relative to a defined lamstatin level range which corresponds to that expressed by a population of individuals corresponding to those from a range of different cohorts.

As detailed herein, embodiments of the invention are applicable to the treatment or prevention of diseases or conditions associated with aberrant lymphatic endothelial cell activity and/or abnormal lymphangiogenesis. Such diseases and conditions include, but are not limited to tumour-induced lymphangiogenesis, cancer metastasis, fibrosis (such as pulmonary fibrosis), lymphangioleiomyomatosis (LAM), rheumatoid arthritis, asthma, transplant rejection, psoriasis or impaired wound repair. The tumour or cancer may be of any type known to occur in or spread via the lymphatic system, including for example thyroid, esophageal, gastric, breast, cervical, lung, pancreatic, endometrial, ovarian, gallbladder, prostate, colorectal and head and neck cancers. The scope of the present disclosure is not intended to be limited by reference to any specific tumour or cancer type. In one exemplary embodiment the tumour is a lung adenocarcinoma.

As would be appreciated, in the context of therapeutic or prophylactic treatment regimens one is generally seeking to downregulate the occurrence of lymphangiogeneis, or lymphatic endothelial cell proliferation or migration. However, in some circumstances it may be desirable to induce or upregulate the occurrence of lymphangiogeneis, or lymphatic endothelial cell proliferation or migration, for example in an in vitro model or an animal model, in order to facilitate an outcome such as providing a system for screening for the effectiveness of adjunctive therapies, prophylactic therapies or for otherwise facilitating the ongoing analysis of diseases and conditions of or pertaining to the lymphatic system. To this end, one may achieve this outcome by decreasing the endogenous lamstatin or tumstatin levels of the subject lymphatic tissue. This may be desirable, for example, in the treatment of conditions such as oedema (such as lymphoedema, surgery-induced oedema or tumour-induced oedema).

It should be understood that the methods of the present invention can be performed either in vitro or in vivo. Although methods are typically to therapeutically or prophylactically treat an individual in vivo in order to achieve a desired clinical outcome, it should nevertheless be understood that it may be desirable that a method of the invention be applied in an in vitro environment, such as in the contexts detailed above. Detection of lymphatic vessel formation and development and the determination of the ability of an agent disclosed herein to inhibit lymphangiogenesis may be performed by any suitable means known to those skilled in the art. By way of example, lymphatic endothelial cell-specific markers such as LyVe-1 may be detected using suitable labelled antibodies.

As defined herein, reference to lamstatin should be understood as a reference to all forms of this molecule and to functional derivatives and homologues thereof. This includes, for example, any isoforms which may arise from alternative splicing of the subject lamstatin mRNA or functional mutants or polymorphic variants of these proteins. The same scope of interpretation is to be afforded to the term tumstatin.

Embodiments of the invention contemplate the administration of lamstatin or tumstatin, or derivatives, variants or homologues thereof. The lamstatin or tumstatin may be derived from humans and may comprise an amino acid sequence as set forth in SEQ ID No: 1 or 3, respectively, or be encoded by a polynucleotide comprising a nucleotide sequence as set forth in SEQ ID NO: 2 or 4, respectively. The lamstatin or tumstatin may be administered as a polypeptide or polynucleotide. The present invention also contemplates the use of derivatives, variants and homologues of human lamstatin and tumstatin.

The polynucleotide may be natural, recombinant or synthetic and may be obtained by purification from a suitable source or produced by standard recombinant DNA techniques such as those well known to persons skilled in the art, and described in, for example, Sambrook et al., Molecular Cloning: a Laboratory Manual, Cold Spring Harbor Laboratory Press (the disclosure of which is incorporated herein by reference). Where a polynucleotide encoding lamstatin or tumstatin is administered, the polynucleotide is typically present in a vector operably linked to suitable regulatory sequences capable of providing for the expression of the coding sequence by a cell. The term “regulatory sequence(s)” includes promoters and enhancers and other expression regulation signals. These may be selected to be compatible with the cell for which the expression vector is designed. Mammalian promoters, such as β-actin promoters and the myosin light chain promoter may be used. However, other promoters may be adopted to achieve the same effect. These alternate promoters are generally familiar to the skilled addressee.

Considering lamstatin by way of example, “derivatives” of lamstatin include functional fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, functionally active regions of the molecule which may be produced by synthetic or recombinant means well known to those skilled in the art. For example suitable derivatives may be peptide fragments such as the peptide fragment comprising the sequence set forth in SEQ ID NO:5, designated CP17 herein.

Derivatives may also be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

The term “variant” as used herein refers to substantially similar sequences. Generally, polypeptide sequence variants possess qualitative biological activity in common. A variant may take any form and may be naturally or non-naturally occurring. A variant polypeptide sequence may be a derivative of a sequence as disclosed herein, which derivative comprises the addition, deletion, or substitution of one or more amino acids. For example, variants of the human lamstatin and tumstatin sequences disclosed herein may possess about 70% sequence identity to the amino acid sequences set forth in SEQ ID Nos: 1 or 3. The variant may comprise amino acid sequences having at least about 75%, 80%, 85%, 90%, 91%, 92%, 93%, 94%, 95%, 96%, 97%, 98% or 99% sequence identity to the amino acid sequences set forth in SEQ ID Nos: 1 or 3. The term “variant” encompasses polypeptide sequences modified from those disclosed herein by any suitable means.

As used herein a “homologue” means that the molecule is derived from a species other than that which is being treated in accordance with the method of the present invention. This may occur, for example, where it is determined that a species other than that which is being treated produces a form of lamstatin which exhibits similar and suitable functional characteristics to that of the lamstatin which is naturally produced by the subject undergoing treatment.

Similarly, the terms derivative, fragment, variant and homologue as used herein may also be applied to nucleotide sequences.

Particular embodiments of the invention contemplate the administration of one or more agents capable of inhibiting or reducing the expression and/or activity of lamstatin or tumstatin. Such inhibitors may directly or indirectly affect lamstatin or tumstatin expression and may act at the level of the lamstatin or tumstatin genes or any products thereof including mRNA (precursor or mature message) or polypeptide. The inhibitor may be a proteinaceous or non-proteinaceous molecule that modulates the transcription and/or translation of the gene or a functional portion thereof (such as a promoter region), or alternatively that modulates the transcription and/or translation of an alternative gene or functional portion thereof, which alternative gene or gene product directly or indirectly modulates the expression of lamstatin or tumstatin. The inhibitory agent may be an antagonist. Antagonists may be any compound capable of blocking, inhibiting or otherwise preventing lamstatin or tumstatin from carrying out their normal biological functions. For the present purposes, the term “antagonist” is used hereinafter to refer to inhibitors of polypeptide activity and expression.

A variety of suitable antagonists may be employed and the scope of the invention is not limited by the selection of any one particular molecule or compound. Suitable antagonists include antibodies, such as monoclonal antibodies, and antisense nucleic acids which prevent transcription or translation of genes or mRNA. Modulation of expression may also be achieved utilising antigens, RNA, ribosomes, DNAzymes, aptamers, antibodies or molecules suitable for use in cosuppression.

Suitable antibodies include, but are not limited to polyclonal, monoclonal, chimeric, humanised, single chain, Fab fragments, and a Fab expression library. Antibodies may act as antagonists of lamstatin or tumstatin polypeptides, or fragments or analogues thereof. Preferably antibodies are prepared from discrete regions or fragments of the polypeptide. Methods for the generation of suitable antibodies will be readily appreciated by those skilled in the art. For example, a suitable monoclonal antibody may be prepared using the hybridoma technology described in Antibodies-A Laboratory Manual, Harlow and Lane, eds., Cold Spring Harbor Laboratory, N.Y. (1988), the disclosure of which is incorporated herein by reference.

Suitable antisense constructs for use in accordance with the present invention include antisense oligonucleotides, small interfering RNAs (siRNAs) and catalytic antisense nucleic acid constructs. Suitable antisense oligonucleotides may be prepared by methods well known to those of skill in the art. Typically oligonucleotides will be chemically synthesized on automated synthesizers. Those skilled in the art will readily appreciate that antisense oligonucleotides need not display 100% sequence complementarity to the target sequence. One or more base changes may be made such that less than 100% complementarity exists whilst the oligonucleotide retains specificity for its target and retains antagonistic activity against this target. Suitable antisense oligonucleotides include morpholinos where nucleotides comprise morpholine rings instead of deoxyribose or ribose rings and are linked via phosphorodiamidate groups rather than phosphates.

An alternative antisense technology, known as RNA interference (RNAi), see, eg. Chuang et al. (2000) PNAS USA 97: 4985) may be used, according to known methods in the art (for example Hammond et al. (2000)Nature 404: 293-296; Bernstein et al. (2001) Nature 409: 363-366; Elbashir et al (2001) Nature 411: 494-498; WO 99/49029 and WO 01/70949, the disclosures of which are incorporated herein by reference), to inhibit the expression or activity of nucleic acid molecules. RNAi refers to a means of selective post-transcriptional gene silencing by destruction of specific RNA by small interfering RNA molecules (siRNA). The siRNA is generated by cleavage of double stranded RNA, where one strand is identical to the message to be inactivated. Double-stranded RNA molecules may be synthesised in which one strand is identical to a specific region of the target transcript and introduced directly. Alternatively corresponding dsDNA can be employed, which, once presented intracellularly is converted into dsRNA. Methods for the synthesis of suitable molecules for use in RNAi and for achieving post-transcriptional gene silencing are known to those of skill in the art.

A further means of inhibiting the expression or activity of lamstatin or tumstatin may involve introducing catalytic antisense nucleic acid constructs, such as ribozymes, which are capable of cleaving lamstatin or tumstatin mRNA transcripts. Ribozymes are targeted to and anneal with a particular sequence by virtue of two regions of sequence complementarity to the target flanking the ribozyme catalytic site. After binding the ribozyme cleaves the target in a site-specific manner. The design and testing of ribozymes which specifically recognise and cleave lamstatin or tumstatin mRNA sequences can be achieved by techniques well known to those in the art (for example Lieber and Strauss, (1995) Mol. Cell. Biol. 15:540-551, the disclosure of which is incorporated herein by reference).

If desired, agents for use in accordance with the present invention may be fused to other compounds, including peptides, polypeptides or other proteinaceous or non-proteinaceous molecules. For example, agents may be fused to molecules to facilitate localisation to the airway tissue.

### Pharmaceutical Compositions

Agents may be administered in accordance with the present invention in the form of pharmaceutical compositions, which compositions may comprise one or more pharmaceutically acceptable carriers, excipients or diluents. Such compositions may be administered in any convenient or suitable route such as by parenteral, oral, nasal or topical routes. In circumstances where it is required that appropriate concentrations of the desired agent are delivered directly to the site in the body to be treated, administration may be regional rather than systemic. Regional administration provides the capability of delivering very high local concentrations of the desired agent to the required site and thus is suitable for achieving the desired therapeutic or preventative effect whilst avoiding exposure of other organs of the body to the compound and thereby potentially reducing side effects.

It will be understood that the specific dose level of a composition of the invention for any particular individual will depend upon a variety of factors including, for example, the activity of the specific agents employed, the age, body weight, general health and diet of the individual to be treated, the time of administration, rate of excretion, and combination with any other treatment or therapy. Single or multiple administrations can be carried out with dose levels and pattern being selected by the treating physician. A broad range of doses may be applicable. Considering a patient, for example, from about 0.1 mg to about 1 mg of agent may be administered per kilogram of body weight per day. Dosage regimens may be adjusted to provide the optimum therapeutic response. For example, several divided doses may be administered daily, weekly, monthly or other suitable time intervals or the dose may be proportionally reduced as indicated by the exigencies of the situation.

Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Pharmaceutical forms suitable for injectable use include sterile aqueous solutions (where water soluble) or dispersions and sterile powders for the extemporaneous preparation of sterile injectable solutions or dispersions. The formulation must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filter sterilisation. Generally, dispersions are prepared by incorporating the various sterilised active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The present invention contemplates combination therapies, wherein agents as described herein are coadministered with other suitable agents that may facilitate the desired therapeutic or prophylactic outcome. For example, in the context of cancer, one may seek to maintain ongoing anti-cancer therapies such as chemotherapy or radiotherapy whilst employing agents in accordance with embodiments of the present invention to inhibit or reduce tumour angiogenesis and/or tumour metastasis. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

The present invention also provides kits suitable for use in accordance with the methods of the invention. Such kits may include for example diagnostic kits for assaying biological samples, comprising an agent for detecting lamstatin or tumstatin, or encoding nucleic acid molecules, and reagents useful for facilitating the detection by the agent(s). Further means may also be included, for example, to receive a biological sample. The agent(s) may be any suitable detecting molecule. Kits according to the present invention may also include other components required to conduct the methods of the present invention, such as buffers and/or diluents. The kits typically include containers for housing the various components and instructions for using the kit components in the methods of the present invention.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

The present invention is further described by reference to the following non-limiting examples.

### EXAMPLES

### Example 1

### Lymphatic Endothelial Cell Viability and Proliferation

To date, an understanding of the function(s) of the NC1 domain of the α5 chain of collagen type IV (lamstatin) have remained elusive. In contrast, roles have been identified for the NC1 domain of the α3 chain of collagen type IV (tumstatin). Tumstatin has been shown, for example, to be anti-angiogenic, to induce apoptosis in proliferating endothelial cells and to be downregulated in airway tissues which have undergone remodelling (see for example co-pending international patent application PCT/AU2007/000106, the disclosure of which is incorporated herein by reference in its entirety).

To determine the function of lamstatin, human primary lymphatic endothelial cells (HMVEC-LLy cells) were exposed to various concentrations of lamstatin and an MTT assay was performed. As a comparison, commercially available tumstatin was used. Specifically, to assess cell viability 4×10³ HMVEC-LLy cells were seeded per well in 100 μl complete EBM-2 media (with bullet kit, Lonza) and incubated for 2 hrs at 37° C., 5% CO2. After this period lamstatin, tumstatin and respective vehicles (1 nM EDTA, pH 3.0) were prepared in complete EBM-2 as serial dilutions in a separate plate and added. Cells were grown for 24 hrs before MTT (1 mg/mL in PBS) was added. After 6 h of further incubation cells were lysed overnight with 10% SDS/0.01 M HCl and measured the following day in a plate reader at 570 nm. The lymphatic cells showed reduced cell viability after 24 hrs with lamstatin only, indicating that this NC domain may exert its main function on the lymphatic system (data not shown).

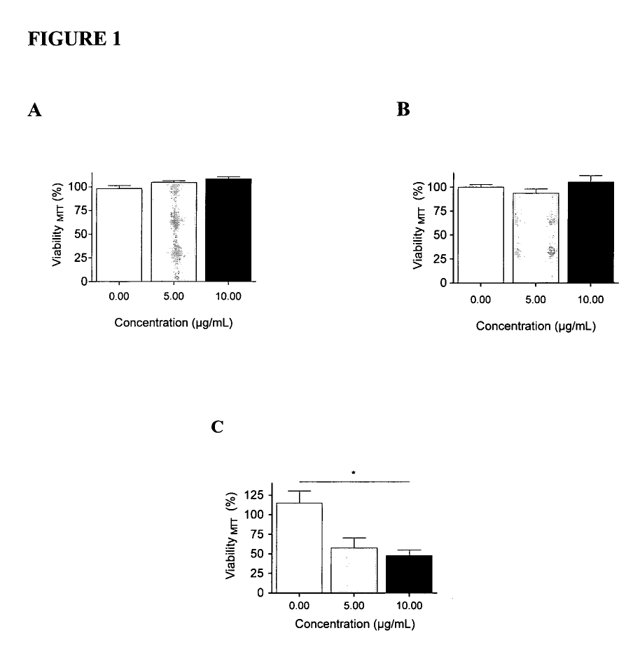

To further delineate this apparent cell specific effect, fibroblasts, epithelial cells and lymphatic cells (HMVEC-LLy) were treated with increasing concentrations of lamstatin. The MTT assay was performed as described above. Fibroblasts and epithelial cells were grown in complete DMEM for 72 hrs before MTT was added. As shown in FIG. 1 only lymphatic endothelial cells responded with decreased cell viability (MTT) after 72 hrs. A baseline activity was measured in the highest concentration of lamstatin, suggesting that the observed effect resembles a cell arresting or anti-proliferative effect, rather then a cytotoxic pathway. To differentiate between antiproliferative and cytotoxic effects, cells were counted 72 hrs after treatment with various concentrations of lamstatin. At a concentration of 10 μg/mL lamstatin, an anti-proliferative effect was observed (data not shown). Viable cells (trypan blue exclusion) were found in all treatment groups, suggesting no cytotoxic effect.

HMVEC-LLy cells were treated with lamstatin and cell attachment and proliferation were measured in realtime using the xCellgenix system (Roche). This system measures a cell index factor, representing impedance mediated by cells. Gold electrodes on the well bottom measure this impedance and the more cells attach to the bottom the higher the impedance (cell index). This measurement allows for detecting early events after treatment such as detachment (e.g. through cell death) or inhibition of proliferation and reduction of cell number.

A Xcellgenix gold coated microtiter plate was seeded with 5×10³ cells (HMVEC-LLy) per well and incubated in 100 μL complete EBM-2 for 24 hrs prior to treatment. In fresh media, serial dilutions of lamstatin or vehicle (1 nM EDTA, pH 3.0) were prepared. Cell culture supernatant was removed and replaced by 100 μL fresh serially diluted lamstatin. The plate was locked into the Xcellgenix recorder and a signal of impedance recorded at 5 min intervals for 6 hrs, and subsequently every 60 min.

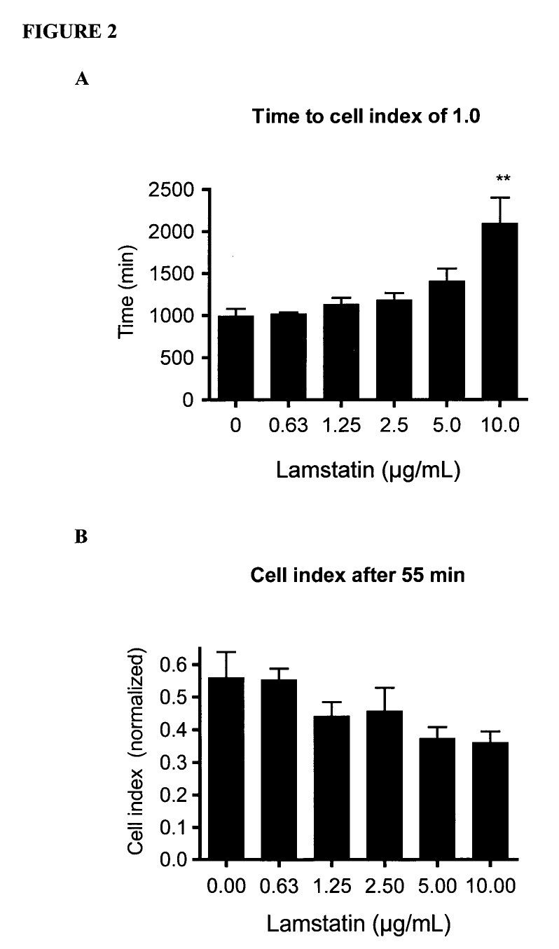

As shown in FIG. 2, cells treated with 10 μg/mL lamstatin required double the amount of time to reach cell number to those in the vehicle control. Additionally, a reduction in cell attachment after 55 min of treatment with lamstatin at various concentrations was observed.

### Example 2

### In Vitro Lymphatic Tube Formation and Cell Migration

Tube formation by HMVEC-LLy cells in the presence of lamstatin, tumstatin and a peptide fragment, designated herein CP17, was assayed.

To assess formation of tubular structures, 48 well plates were coated with 130 μL Matrigel® (BD Bioscience), which was solidified at 37° C. over 45 min. 2×10⁴ HMVEC-LLy cells in complete EBM-2 (500 μL) were seeded per well and allowed to attach for 30 min, before lamstatin, tumstatin, CP17 or vehicle was added to the wells. Photos were taken with a Kodak Digital Camera every 2 hrs. Tube formation peaked at 24 hrs, at which time tubular structures were further analysed to determine length (in pixels) and number of tubes per high power field (HPF) area (px²). The HPF was divided in nine regions of equal size and tubes per region were counted. Mean and SEM were calculated and plotted.

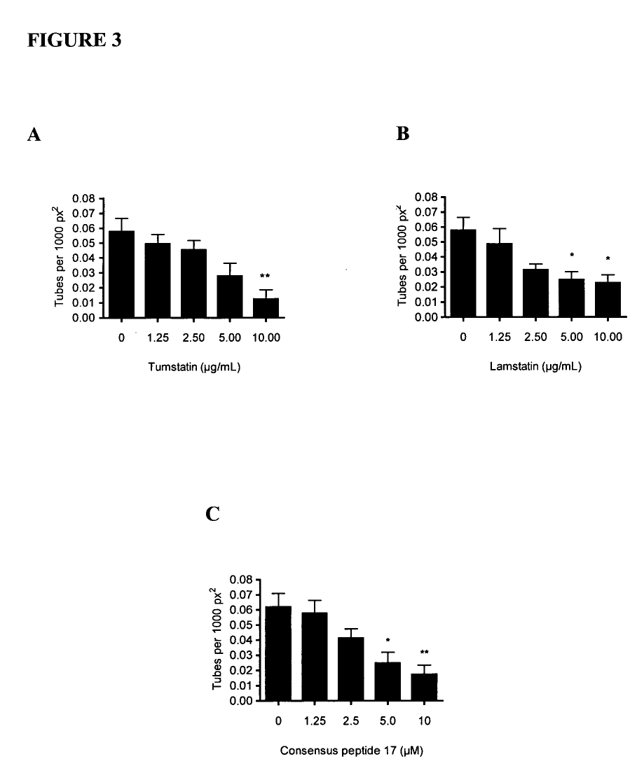

The date provided in FIG. 3 suggest a role for lamstatin, tumstatin and functional peptide fragments thereof in inhibiting new lymphatic vessel formation (lymphangiogenesis).

VEGF-D has been shown to promote lymphangiogenesis and lymphatic metastasis in tumours (Stacker et al. Nature Med. 7, 186-191, 2001). The inventors therefore investigated the ability of HMVEC-LLy cells to migrate towards a VEGF-D gradient.

FluoroBlok BioCoat 24-well inserts (BD Bioscience) were seeded with 4×10⁴ HMVEC-LLy cells in 750 μL DMEM with 0.1% BSA, 1% antibiotics and 25 mM HEPES and incubated for 1 hr. The bottom well was filled with 500 μL medium (as described above) with or without 10 ng/mL VEGF-D (R&D Systems) as well as various concentrations of lamstatin, tumstatin, CP17 or vehicle. Additionally, top wells were loaded with inhibitors to match the bottom well concentration. According to manufacturer's instructions cells were allowed to migrate for 24 hrs before bottom media was removed. Thereafter the underside of the transwell with the migrated cells was washed twice with HANKS buffer and placed in a 4 μg/mL calcein (HANKS) solution for 90 min at 37° C. Signal was read with a bottom read fluorescence microplate reader at 485/515 nm (excitation/emission).

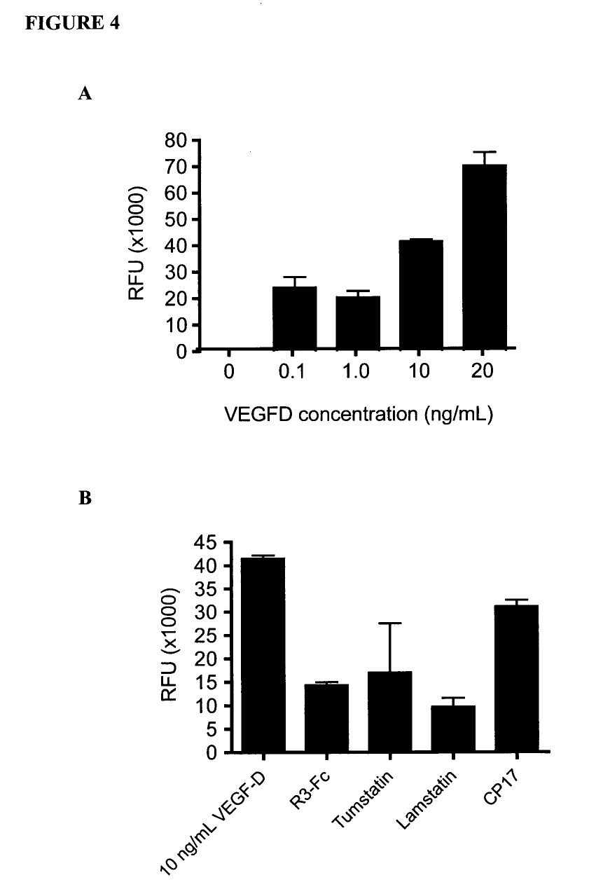

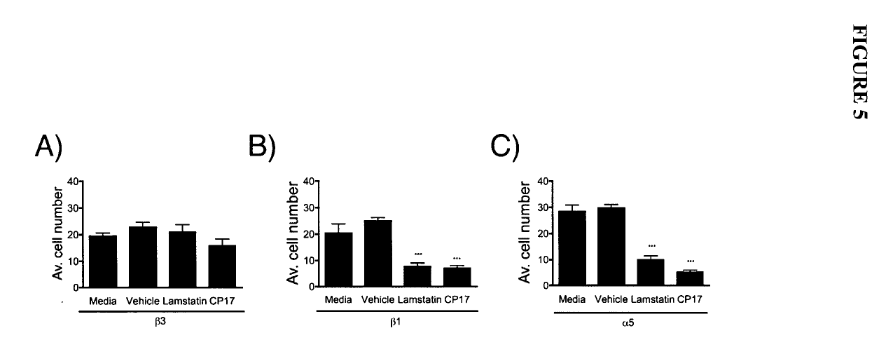

As shown in FIG. 4A increasing concentrations of VEGFD attract lymphatic endothelial cells in a concentration related manner. The addition of a VEGFD specific blocking fusion receptor (R3-FC) diminished migration as expected (FIG. 4B). Lamstatin was equally potent in blocking cell migration (FIG. 4B) and tumstatin showed similar tendencies. Interestingly, while both lamstatin and tumstatin were shown to significantly inhibit cell migration, the same effect was not observed in cells exposed to peptide CP17.

### Example 3

### Integrin Binding to Lamstatin

The attachment of cells to surrounding cells or molecules in the extracellular matrix is mediated by cell surface receptors called integrins. The inventors investigated the identity of the integrins involved in the binding of lymphatic endothelial cells to lamstatin and peptide CP17 in vitro.

Antibodies for integrin α5 (MAB1956Z; MAB1953Z), integrin β1 (MAB2253Z) and integrin β3 (MAB1957Z) (all Millipore, USA) were coated to 96 well tissue culture plates (Nunc, USA) at a concentration of 10 μg/mL overnight at 4° C. in sterile coating buffer (50 mM Na2CO3, 50 mM NaHCO3, pH 9.6). Plates then were blocked with sterile 1% BSA/PBS for 1 h at 37° C. and washed twice with EBM-2 MV (Lonza, Basel, Switzerland). MMVEC-LLy cells were carefully removed from culture flasks with non-enzymatic detachment solution (Trevigen, Md., USA) and washed twice with EBM-2 (Lonza, Basel, Switzerland). Volume was adjusted to a cell concentration of 50,000 cells/100 μL and divided into aliquots for treatment with recombinant lamstatin (10 μg/ml), CP17 (5 μM) or vehicle (1 nM EDTA, pH 3.5) only. Cells were then transferred to the antibody-coated plates and incubated for 1 h at 37° C. Wells were then washed with HANKS twice to remove unbound cells and fixed with 4% paraformaldehyde (in PBS) for 15 min at room temperature. Wells thereafter were photographed with an Olypmus CAMEDIA C-4000 camera mounted to an Olympus CK2 microscope. Pictures were divided into 25 segments and cells of 5 center-near quadrants were counted and averaged. As shown in FIG. 5, monoclonal antibodies against integrins β1 (FIG. 5B) and α5 (FIG. 5C) significantly inhibited cell binding to lamstatin and CP 17, whereas a monoclonal antibody against integrin β3 (FIG. 5A) had no effect on binding. These results demonstrate that integrins β1 and α5, but not β3, are involved in lymphatic endothelial cell binding to lamstatin. Using a similar approach, the inventors observed no expression of integrin α4 on the surface of HMVEC-LLy cells (data not shown).

The inventors have determined that the predominant integrins that mediate the binding of lymphatic endothelial cells (HMVEC-LLy cells) in vitro to lamstatin, tumstatin and peptide CP17 (integrins α5 and β1) are different to those integrins involved in the binding of umbilical vein endothelial cells (HUVECs) to lamstatin, tumstatin and peptide CP17 (data not shown). These data suggest fundamentally different roles, and mechanisms by which activity is mediated, for lamstatin and tumstatin in endothelial cells of the lymphatic system compared to other endothelial cells.

### Example 4

### Lymphangioleiomyomatosis (LAM) and Lamstatin

Lymphangioleiomyomatosis (LAM) is a disease of the lungs that appears to be associated with abnormal proliferation of smooth muscle cells. This underlies the formation of characteristic LAM nodules in the lung. These pulmonary nodules are responsible for cystic destruction of the lung, recurrent pneumothoraces and a steady decline in pulmonary function.

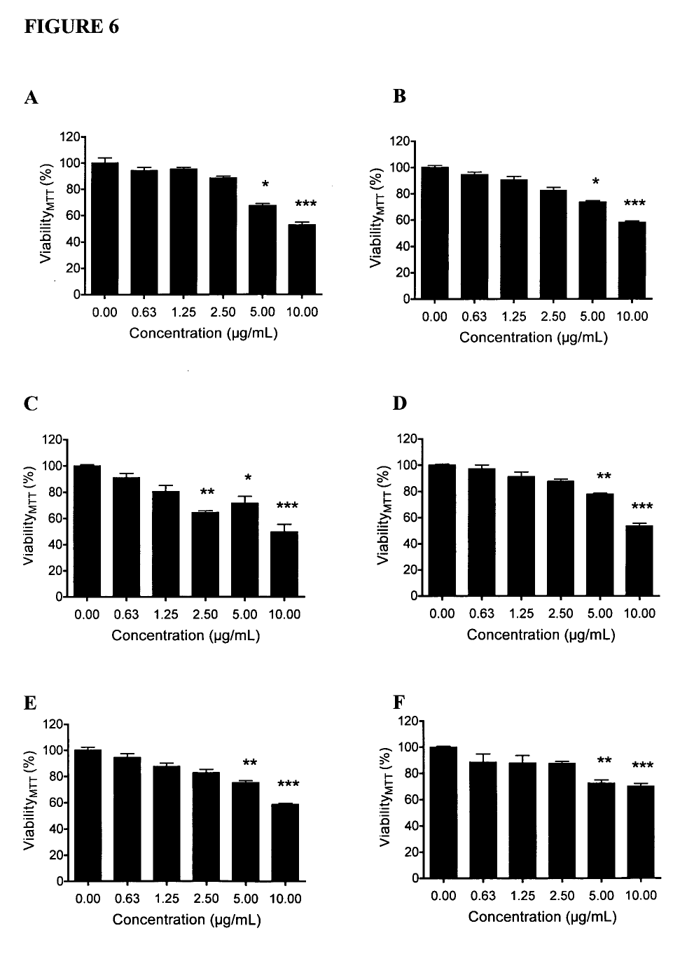

The inventors isolated lung cells from a LAM patient, which cells originated from the LAM nodules. These cells were termed according to their location within the nodule, as intermediate layer in the nodule, centre cells and outer nodule cells. These cells were then treated with lamstatin or tumstatin and cell viability measured after 72 hrs. The MTT viability assay was performed as described above in Example 1. As shown in FIG. 6, lamstatin was effective in reducing cell viability of all nodule cell types at both high (e.g. 10 μg/mL) and low (1.25-2.5 μg/mL) concentrations. Tumstatin was also effective in reducing cell viability at higher concentrations.

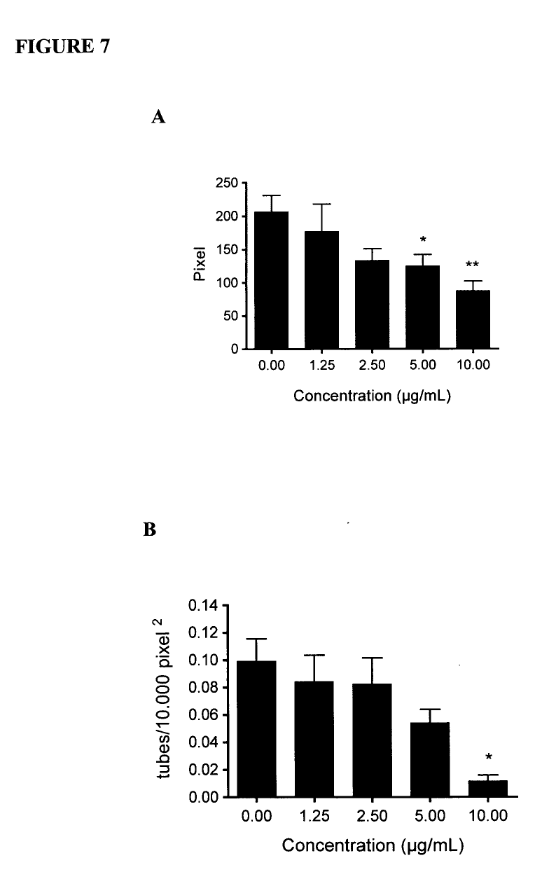

The inventors then analysed tube formation in LAM nodule cells and found only the outer nodule cells responded with pseudo-tube formation. Assay for tube formation was performed as described above in Example 2. Photos were taken at 2, 3, 4, 5 and 24 hrs after seeding. Lamstatin was added directly after seeding to inhibit prior tube formation. Tube formation peaked at 5 hrs and after 24 hrs no tubular structures were observed (both treated and untreated) (data not shown). Tube length and numbers of tubular structures per square pixel for each photo were determined as shown in FIG. 7. At 5hrs tube formation, as measured by both tube length and tube number, was significantly inhibited by lamstatin.

### Example 5

### In Vivo Inhibition of Tumour-Induced Lymphangiogenesis

A mouse ear tumour plug model was utilized in which green fluorescent protein (GFP)-tagged lung adenocarcinoma cells (LNM35 AAV-GFP) were injected between the two layers of skin of the ears of NOD/SCID mice. 1×10⁶ tumour cells were embedded in 50 pt growth factor free Matrigel® medium enriched with vehicle (1 nM EDTA, pH3.0) or lamstatin at either 10 μg/mL or 100 μg/mL. Mice were divided into three groups.

Group 1 (n=9): One ear of each mouse injected with tumour cells+10 μg/mL lamstatin; the other ear injected with tumour cells+vehicle control.
Group 2 (n=9): One ear of each mouse injected with tumour cells+100 μg/mL lamstatin; the other ear injected with tumour cells+vehicle control.
Group 3 (n=3): Each ear injected with Matrigel® only.

Injections were single shots such that at least 25 μL was injected. 12 days after injection were sacrificed by anesthetisation and cervical dislocation. Ears were removed for whole mount staining. Flaps of skin from each ear were carefully separated, fixed and stained for either the lymphatic endothelial cell specific marker LyVe-1 (rabbit polyclonal anti-LyVe-1 antibody detected using anti-rabbit Alexafluor 647) or the vascular endothelial cell specific marker PECAM (CD31 rat anti-mouse antibody clone MEC13.3 (Pharmingen), detected with anti-mouse Alexafluor 594). Tumour cells were visualised by detection of GFP expression. Images of representative regions of ears were obtained using a confocal microscope and LyVe-1 or PECAM staining was quantified.

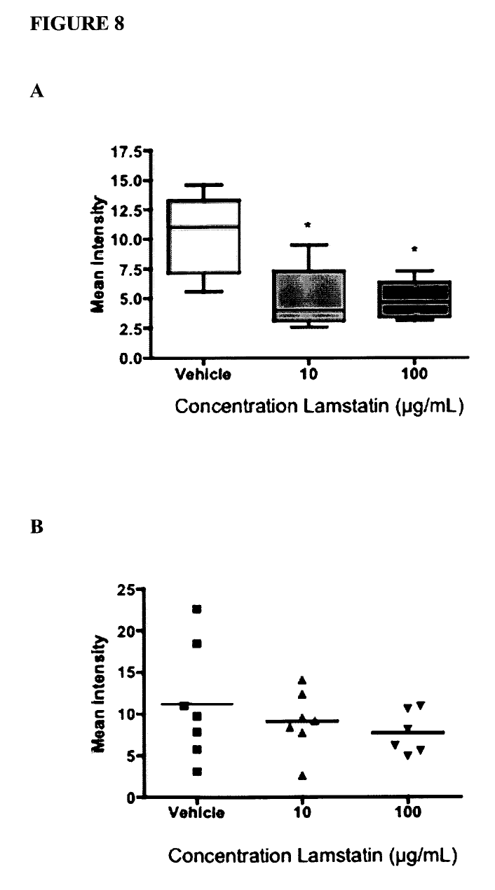

As shown in FIG. 8A mean intensity of LyVe-1 staining was significantly reduced (by approximately 60% compared to vehicle only) in the case of tumour cell+lamstatin injection (both 10 μg/mL and 100 μg/mL lamstatin). In contrast, no obvious reduction in PECAM staining was observed in the vicinity of tumour cells in the presence of 10 μg/mL or 100 μg/mL lamstatin (FIG. 8B). These results indicate lymphatic endothelial cell-specific inhibitory effect of lamstatin in the vicinity of the tumour in vivo.

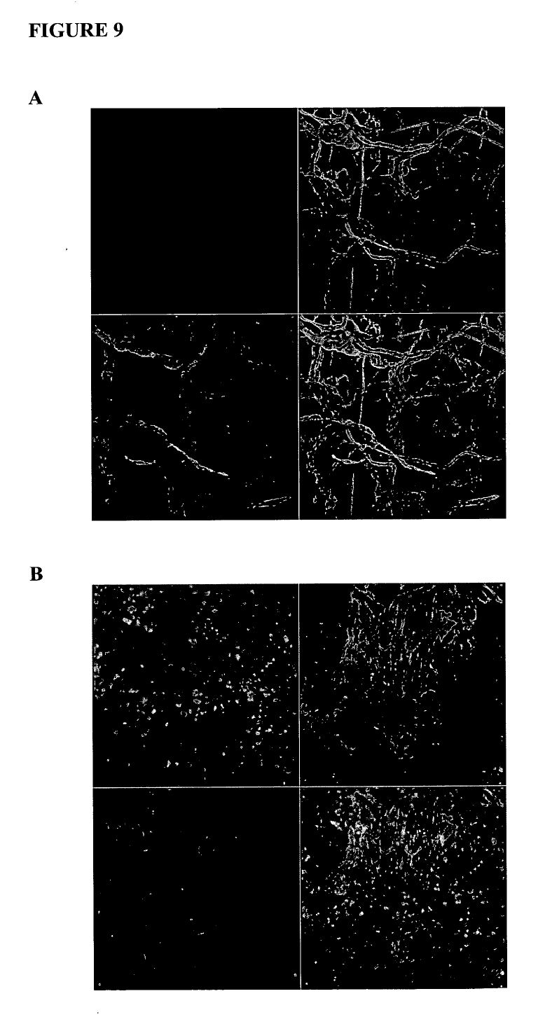

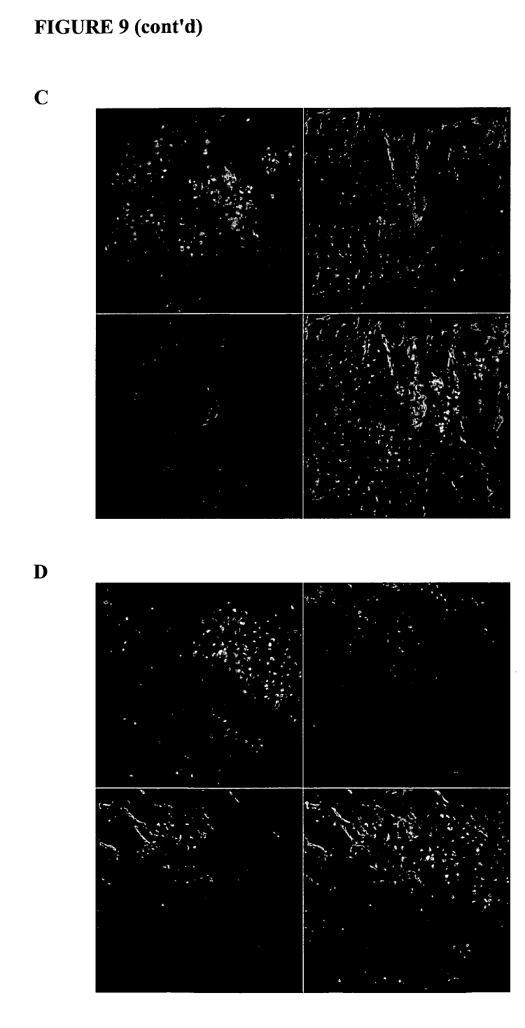

These data are supported by visual inspection of lymphatic vessel formation and blood vasculature around the tumour (FIG. 9). In the case of mice receiving Matrigel® medium only blood and lymphatic vasculature were observed to develop normally with blunt end and no tips extending or connecting to each other (FIG. 9A). Tumour-induced blood and vasculature development is shown in FIG. 9B. A thin network of intertwined and interconnected lymphatic vessels was observed, with some tips visible. In animals injected with 10 μg/mL lamstatin a reduction in the number of lymphatic vessels and a dilation of lymphatic vessels was observed (FIG. 9C). In animals injected with 100 μg/mL lamstatin a further reduction in lymphatic vessel network density was observed compared to those animals that received 10 μg/mL lamstatin, with fewer regions per specimen showing strong tumour induced lymphatic vasculature (FIG. 9D). In contrast to the clear changes in lymphatic vasculature development observed, lamstatin had no discernable effect on the development of blood vasculature around the tumour (FIG. 9B to 9D).

Using the LyVe-1 whole mount stained mouse ear sections, the inventors further investigated the morphology of lymphatic vessel formation. Specifically confocal images were loaded into Image J software (www.sbweb.nih.gov/ij/), a 5×5 grid overlayed and vessels examined for branching and looping. The functionality of a lymphatic network is based on its maturity and in general it is accepted that a tumour induces a rather immature network formation. Reconnecting vessels (measured as loops) and increased branching (measured as branches) are signs of immature vessels induced by, for example, a tumour (see for example Shayan et al. Growth Factors 25, 417-425, 2007).

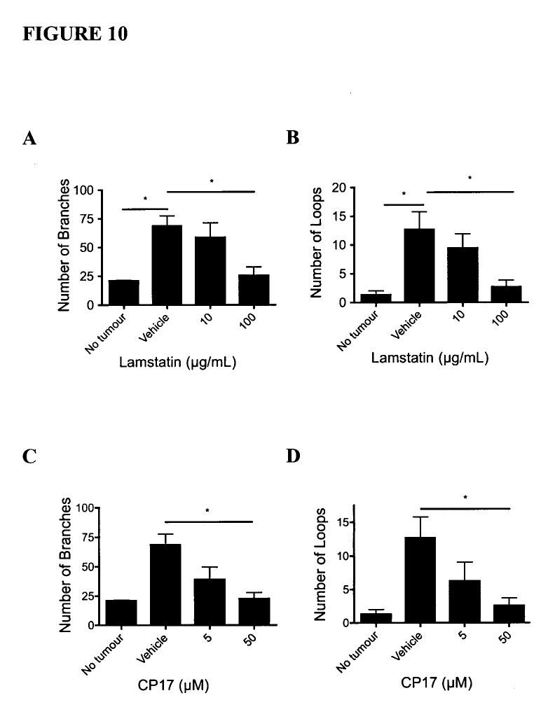

Branches were defined as two clearly distinguishable vessels that separate out without rejoining. Loops were defined as small, circular vessel structures in the same focal plane. Branches or loops were counted separately per image and mean values were calculated for each treatment group. Both lamstatin (FIGS. 10A and 10B) and CP17 (FIGS. 10C and 10D) significantly reduced the number of branches and loops to baseline levels. These results indicate that lamstatin and its peptide CP17 are able reverse the effects of the tumour in terms of excessive lymphangiogenesis.

1. A method for inhibiting proliferation and/or migration of lymphatic endothelial cells in a subject, the method comprising administering to the subject, or to lymphatic endothelial cells derived therefrom, an effective amount of a collagen type IV-derived NC1 domain polypeptide or a fragment, derivative or variant thereof, a polynucleotide encoding the same, or an agent capable of increasing the expression or production of the NC1 domain polypeptide.

2. The method of claim 1 wherein the collagen type IV-derived NC1 domain polypeptide is the NC1 domain of the α5 chain of collagen type IV (lamstatin).

3. The method of claim 2 wherein the lamstatin comprises an amino acid sequence as set forth in SEQ ID NO:1.

4. The method of claim 2 wherein the lamstatin is encoded by a polynucleotide comprising a nucleotide sequence as set forth in SEQ ID NO:2.

5. The method of claim 1 wherein the collagen type IV-derived NC1 domain polypeptide is the NC1 domain of the α3 chain of collagen type IV (tumstatin).

6. The method of claim 5 wherein the tumstatin comprises an amino acid sequence as set forth in SEQ ID NO:3.

7. The method of claim 5 wherein the tumstatin is encoded by a polynucleotide comprising a nucleotide sequence as set forth in SEQ ID NO:4.

8. The method of claim 1 wherein the fragment comprises the amino acid sequence VCNFASRNDYSYWLSTP (SEQ ID NO:5) or a variant thereof.

9. A method for inhibiting lymphangiogenesis, the method comprising administering to a subject, or cells or lymphatic tissue derived therefrom, a collagen type IV-derived NC1 domain polypeptide or a fragment, derivative or variant thereof, a polynucleotide encoding the same, or an agent capable of increasing the expression or production of the NC1 domain polypeptide.

10. The method of claim 9 wherein the subject suffers from, or is predisposed to, a disease or condition associated with excessive or otherwise aberrantly regulated lymphangiogenesis.

11. The method of claim 9 wherein the lymphangiogenesis is tumour-induced lymphangiogenesis.

12. The method of claim 9 wherein cancer metastasis is reduced following the administration step.

13. A method as in claim 1 for the treatment or prevention of a disease or condition associated with aberrant lymphatic endothelial cell activity, the method comprising administering to the subject, or to lymphatic endothelial cells derived therefrom, an effective amount of a collagen type IV-derived NC1 domain polypeptide or a fragment, derivative or variant thereof, a polynucleotide encoding the same, or an agent capable of increasing the expression or production of the NC1 domain polypeptide.

14. The method of claim 13 wherein the disease or condition is associated with abnormal, excessive or otherwise aberrantly regulated lymphangiogenesis.

15. The method of claim 13 wherein the disease or condition is selected from, for example, tumour-induced lymphangiogenesis, cancer metastasis, fibrosis, lymphangioleiomyomatosis (LAM), rheumatoid arthritis, asthma, transplant rejection, psoriasis, impaired wound repair, lymphangioma or Kaposi's sarcoma.

16. The method of claim 15 wherein the fibrosis is pulmonary fibrosis.

17. (canceled)

18. (canceled)

19. A method for the promotion of lymphangiogenesis, the method comprising administering to a subject, or cells or lymphatic tissue derived therefrom, an inhibitor of a collagen type IV-derived NC1 domain polypeptide or a fragment, derivative or variant thereof, a polynucleotide encoding the same.

20. The method of claim 19 wherein the subject suffers from a condition characterised by, or otherwise associated with, impaired lymphangiogenesis.

21. The method of claim 20 wherein the condition is oedema.

22. The method of claim 21 wherein the oedema is lymphoedema, surgery-induced oedema or tumour-induced oedema.
